(19) United States
(12) Patent Application Publication
(10) Pub. No.: US 2014/0271509 A1
(43) Pub. Date: Sep. 18, 2014

(54) Compositions for Skin Lightening and/or Brightening

(71) Applicant: INDERMA, INC., Mill Valley, CA (US)

(72) Inventors: Eva Claiborne, San Rafael, CA (US); Jennifer Clara Ellis, Lawton, OK (US)

(73) Assignee: INDERMA, INC., Mill Valley, CA (US)

(21) Appl. No.: 13/841,479

(22) Filed: Mar. 15, 2013

(51) Int. Cl. A61K 8/97; A61K 8/66; A61K 8/73; A61Q 19/08; A61K 8/67; A61K 8/92; A61Q 19/02; A61K 8/64; A61K 8/60

(52) U.S. Cl. CPC A61K 8/97; A61K 8/64; A61K 8/66; A61K 8/73; A61K 8/602; A61K 8/678; A61K 8/92; A61Q 19/02; A61Q 19/08
USPC 424/62

(57) ABSTRACT

This disclosure describes compositions for skin lightening, brightening, whitening, reducing the appearance of age spot and/or improving the appearance of skin. The composition may include two or more tryosinase inhibiting compounds and one or more extracts from at least one plant species to inhibit alpha-Melanoctye-stimulating hormone. Other embodied compositions include at least one peptide having tyrosinase inhibiting qualities; at least on hydroquinone derivative; and at least one extract from at least one plant species.

### BACKGROUND

Multiple cosmetic treatments exist to enhance an individual's natural appearance, skin condition and health. For example, various skin care products are routinely applied to an individual's face, neck, hands or other areas of the body to minimize the appearance of aging or melasma (e.g., minimize dark spots or age spots). However, these skin care products generally contain only the smallest effective amount of one active ingredient due to the extreme cost of available active ingredients. As such, existing skin care products only achieve minimal results over an extended period of time. Additionally, as life expectancies continue to rise and people continue to live longer, there is a continued increase in the demand for treatments which reverse the appearance of aging. As the demand for such cosmetic treatments continue to increase, the skin care and cosmetic market also continues to grow. Accordingly, there remains a need in the art from improved cosmetic treatments that provide improved skin appearance by, for example, minimizing the appearance of dark spots or age spots associated with aging.

### BRIEF SUMMARY

This summary is provided to introduce simplified concepts of methods and compositions for reducing the appearance of aging and improving the condition of skin, such as skin covering the face, neck, shoulders, arms, back, chest or other body area. Additional details of example methods and compositions are further described below in the Detailed Description. The embodiments described herein are not mutually exclusive and aspects of the various embodiments may be combined to arrive at other embodiments within the scope of the claims. This summary is not intended to identify essential features of the claimed subject matter, nor is it intended for use in determining the scope of the claimed subject matter.

According to an embodiment, the present invention concerns a composition comprising two or more tryosinase inhibiting compounds; one or more extracts from at least one plant species to inhibit alpha-Melanoctye-stimulating hormone; and one or more cosmetically acceptable carriers.

According to another embodiment, the invention concerns a composition comprising at least one peptide having tyrosinase inhibiting qualities; at least on hydroquinone derivative; at least one extract from at least one plant species; and a cosmetically acceptable carrier.

### BRIEF DESCRIPTION OF THE DRAWINGS

The detailed description is set forth with reference to the accompanying figure.

FIG. 1 shows photographs of a subject before (on the viewer's left) and after (on the viewer's right) a first treatment with a composition according to the present invention. The pictures show a post treatment reduction in dark spot and blotches on the subject's skin.

FIG. 2 shows additional photographs of the subject before and after treatment with a composition according to the present invention. These pictures also show a post treatment reduction in dark spot and blotches. Furthermore, these pictures show a post treatment improvement in the brightness and pigment uniformity of the subject's skin.

### DETAILED DESCRIPTION

The present disclosure relates to compositions which may be used on skin (e.g., the skin of a user or the skin of a subject in need of treatment) to reduce the appearance of dark spot, age spots and improve the overall appearance of skin. According to an embodiment, the compositions may be used as compositions to brighten the skin, even out skin tone and/or reduce the appearance of age spots. According to an embodiment, the composition may comprising of two of more active ingredient where each active ingredient provides a separate, safe and effective mechanism for reducing the appearance of dark spot, age spots and improving the overall tone and consistency of skin

According to an embodiment, the present invention concerns a composition comprising two or more tryosinase inhibiting compounds; one or more extracts from at least one plant species to inhibit alpha-Melanoctye-stimulating hormone; and one or more cosmetically acceptable carriers. Furthermore, one or more of the composition ingredients are present in effective amounts for preventing, reducing the number and/or appearance of dark spot or age spot (e.g., reducing the appearance of melanin, a dark skin pigment) on skin.

According to certain embodiments, the tyrosinase inhibiting compounds may be one or more peptides, a hydroquinone derivative, one or more anti-oxidants, or a combination thereof. The one or more peptides may be from a natural and/or synthetic source. For example, the peptides may be one or more of Glutathione, Glutathione-Reductase, Dimethyl Methoxy Chromanyl Palmitate or a combination thereof. However, other peptides are envisioned. It is well known in the art that skin hyperpigmentation has been attributed to the formation of melanin, a dark pigment formed by the amino acid, tyrosine within melanocytes. The conversion of tyrosine to melanin within the melanocytes is mediated by the enzyme tyrosinase. Therefore, peptides that are effective tyrosinase inhibitors may inhibit the formation of melanin and thus may reduce the appearance of undesirable skin hyperpigmentation. Furthermore, in some instance, application of such peptides may lead to a de-pigmenting effect on skin. As a result, the skin may appear with fewer blotches, discolorations, age spots and have lightened, brightened or whitened appearance.

Typical compositions of the invention contain from about 0.01% to about 0.1% by weight, from about 0.2% to about 0.5% by weight, from about 0.6% to about 1.0% by weight, by weight of the peptides. Lower concentrations may be employed for less pronounced conditions (e.g., situations where there skin has fewer age spot and/or lighter age spots) and higher concentrations may be employed with more acute conditions (e.g., situations where there skin has more age spot and/or darker age spots).

According to certain embodiments, the hydroquinone derivative may be arbutin. Arbutin is a glycosylated hydroquinone which may be isolated from the leaves of a bearberry scrub, a cranberry, a blueberry, and/or a pear. Arbutin may act to inhibit tyrosinase and, thus, prevent the development of melanin as described above. Arbutin may be introduced to the composition by itself or within a compound in combination with other ingredients.

According to certain embodiments, the one or more anti-oxidants comprise linolenic acid, tocopheryl Acetate, Olea Europaea fruit oil, Ascorbyl palmitate or a combination thereof. Anti-oxidant may act to inhibit the oxidation of tyrosinase. Limiting the ability of tyrosinase to oxidize may prevent the chemical reaction or cascading effect producing melanin in the melanocytes. Typical compositions of the invention contain from about 0.01% to about 0.1% by weight, from about 0.2% to about 0.5% by weight, from about 0.5% to about 1.0% by weight, by weight of the anti-oxidants.

According to certain embodiments, the one or more plant species extracts to inhibit alpha-Melanoctye-stimulating hormone comprises at least one of: Hydrolyzed Beta maritima Extract and/or Lepidium Sativum Sprout Extract. Hyrdolyzed Beta maritima Extract is the hydrolysate of the extract of the whole plant, Beta maritime L., Chenopodiaceae (Sea Beet). Lepidium Sativum Sprout Extract is an extract of garden cress spouts that has been found to exhibit a strong skin whitening effect. Lepidium Sativum Sprout Extract, as incorporated into a liposomal preparation, inhibits pigmentation by targeting the neutralization of reactive oxygen species and the inhibition of alpha-Melanocyte-stimulating hormone (α-MSH). α-MSH is a naturally occurring peptide hormone that is vital to upstream reaction steps in the melanin enzymatic cascade that stimulates skin pigmentation. Lepidium Sativum Sprout Extract may be introduced to the composition by itself or within a compound in combination with other ingredients (e.g., within a liposomal preparation).

According to certain embodiments, the composition may further include stem cells from at least one plant species. For example, the stem cells may be from a grape vine, a rose plant, an apple tree, an argan tree or the stem cells may be a combination of stem cells from two or more of those plants. In certain embodiments, the stem cell may be Malus Domestica Fruit Cell Culture Extract which has demonstrated an ability to protect the longevity of human stem cells. Stem cells from plant species are undifferentiated cells located in root meristem, shoot meristem, and vascular meristem which create differentiated cell types and self-renew. Typical compositions of the invention contain from about 0.01% to about 0.05% by weight, from about 0.06% to about 0.1% by weight, from about 0.15% to about 0.5% by weight, or from about 0.5% to about 1.0% by weight of the stem cells. Lower concentrations may be employed for less pronounced conditions (e.g., situations where there skin has fewer age spot and/or lighter age spots) and higher concentrations may be employed with more acute conditions (e.g., situations where there skin has more age spot and/or darker age spots).

According to certain embodiments, the composition may include at least one extract from at least one plant species in addition or alternative to the plant stems cell extracts listed above. For example, in some embodiments, the composition may include Glycyrrhiza Uralensis (Licorice) Root Extract and/or Bellis Perennis (Daisy) Flower Extract. Licorice Root Extract has been demonstrated to strengthen the immune system and has skin whitening effects. Daisy Flower Extract has been demonstrated to sooth and restore inflamed and/or irritated skin. Typical compositions of the invention contain from about 0.1% to about 0.5% by weight, from about 0.5% to about 0.9% by weight, from about 1.0% to about 1.05% by weight, by weight of Glycyrrhiza Uralensis (Licorice) Root Extract.

The compositions may include additional ingredient or cosmetically acceptable carriers such as, for example, surfactants (e.g., Decyl Glucoside, Laureth-7), emulsifiers (e.g., Cetyl Alcohol, Ceteareth-20), foam modulators (e.g., Cocamide MEA), viscosity modifiers (Disodium EDTA, Cetyl Alcohol), humectants (E.g., Hyaluronic acid, caprylyl Glycol), diluents (e.g., water), fillers, pH modifying agents (e.g., Sodium Hydroxide, trithanolamine), colorants, solvents (e.g., purified water), preservatives (e.g., Phenoxyethanol, Methylisothiazolinone, Methylchloroisothiazolinone, Metyldibromo Glutaronitrile), conditioners (e.g., Lecithin, Caprylic, Capric Triglyceride, Butylene Glycol), thickeners (e.g., Carbomer, Cetyl Alcohol, C13-14 Isoparaffin), perfumes (e.g., derived from synthetic ingredients flowers, herbs or organic esters), emollients (e.g., apricot kernel oil, Simmondsia Chinensis (Jojoba) seed oil, Olea Europaea (Olive) fruit oil), vitamins (Tocopheryl acetate (Vitamin E), Linoleic Acid (Vitamin F), Ascorbyl Palmitate (Vitamin C)), minerals, and/or moisturizers (e.g., glycerin, phospholipids, ceramides, sphingolipids).

The compositions may be formulated into one of various mediums or delivery systems for application of the composition to skin such as a serum, cream, gel, emulsion, spray, powder, lotion, ointment, soap, or stick.

Typically, topical application to skin sites is accomplished in association with one or more cosmetically acceptable carrier, and particularly one in which the active ingredient is soluble per se or is effectively solubilized (e.g., as an emulsion, microemulsion or liposomal preparations). Where employed, the carrier is inert in the sense of not bringing about a deactivation or oxidation of active or adjunct ingredient(s), and in the sense of not bringing about any adverse effect on the skin areas to which it is applied. For example, the compositions according to the present invention are applied in admixture with a dermatologically acceptable carrier or vehicle (e.g., as a serum, cream, gel, emulsion, spray, powder, lotion, ointment, soap, stick, or the like) so as to facilitate topical application and, in some cases, provide additional beneficial effects as might be brought about, e.g., by moisturizing, firming, conditioning, calming and/or scenting of the affected skin areas. While the carrier for dermatological/cosmetic compositions can consist of a relatively simple solvent or dispersant such as water, other suitable carriers comprise a composition more conducive to topical application. In particular, a dermatological composition of some embodiments will form a film or layer on the skin to which it is applied so as to localize the application and provide some resistance to washing off by immersion in water or by perspiration and/or aid in the percutaneous delivery of the active agent. Many preparations are known in the art, and include lotions containing oils and/or alcohols and emollients such as olive oil, hydrocarbon oils and waxes, silicone oils, other vegetable, animal or marine fats or oils, glyceride derivatives, fatty acids or fatty acid esters or alcohols or alcohol ethers, lecithin, lanolin and derivatives, polyhydric alcohols or esters, wax esters, sterols, phospholipids and the like, and generally also emulsifiers (nonionic, cationic, or anionic), although some of the emollients inherently possess emulsifying properties. These same general ingredients can be formulated into a cream rather than a serum, or into gels, or into solid sticks by utilization of different proportions of the ingredients and/or by inclusion of thickening agents such as gums or other forms of hydrophilic colloids. Such compositions are referred to herein as dermally, dermatologically, or cosmetically acceptable carriers.

Generally in the practice of methods of the invention, the composition is topically applied to skin in need of skin lightening, brightening and/or whitening treatment in a predetermined or as-needed regimen either at intervals by application of a serum or the like, it generally being the case that gradual lightening, brightening or whitening is noted with each successive application. According to a certain embodiment, the methods include applying a composition according to present invention in one form (e.g. a serum) followed by applying the same or a different composition according to the present invention in another form (e.g. a cream).

“Cosmetically effective amount” or “effective amount” refers to the amount of a compound that, when administered to a subject for treating a cosmetic condition, or at least one of the symptoms of the condition, is sufficient to affect such treatment for the condition, or symptom. The “cosmetically effective amount” can vary depending on the compound, the condition and/or symptoms of the condition, severity of the condition and/or symptoms of the condition, the age of the subject to be treated, and/or the weight of the subject to be treated. An appropriate amount in any given instance can be readily apparent to those skilled in the art or capable of determination by routine experimentation.

“Treating” or “treatment” of any condition or symptom refers to arresting or ameliorating the condition, or at least one of the clinical symptoms of a condition, reducing the risk of acquiring condition or at least one of the clinical symptoms of a condition, reducing the development of a condition or at least one of the clinical symptoms of the condition, or reducing the risk of developing a condition or at least one of the clinical symptoms of the condition. “Treating” or “treatment” also refers to inhibiting the condition, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying or preventing the onset or reoccurrence of the condition or at least symptoms thereof in a subject which may be exposed to or predisposed to or may have previously suffered from a condition even though that subject does not yet experience or display symptoms of the condition.

It should be understood that the ingredients particularly mentioned above are merely examples and that some embodiments of formulations comprising the compositions of the present invention include other suitable components and agents. The compositions of the invention may be used for, among other things, pharmaceutical and cosmetic purposes and may be formulated with different ingredients according to the desired use.

### EXAMPLES

Certain embodiments of the present invention now will be illustrated by the following Examples. The present invention may, however, be embodied in many different forms and should not be construed as limited to the embodiments set forth herein; rather, these embodiments are provided so that this disclosure will be thorough and complete, and will fully convey the scope of the invention to those skilled in the art.

### Example 1

In this example, a subject was treated with a composition according the present disclosure. Before and after photographs are included as FIGS. 1 and 2.

First, the subject's face was cleansed with a cleanser.

After the cleanser was removed with a wet towel, a peptide crystal exfoliating composition was applied to the subject's face with care taken to avoid the eye area. A chemical exfoliating composition was then applied to the subject's face on top of the peptide crystal exfoliation composition. Both the peptide crystal exfoliating composition and the chemical exfoliating composition were left on the subject's face for about 5-10 minutes wherein a white foam appeared on the subject's face. Both composition were then removed a wet towel.

The subject's face was then cleansed to remove any residual peptide crystal exfoliation composition and chemical exfoliating composition with the cleanser described above.

A toner was then sprayed on the subject's face to neutralize and remove any traces of the cleanser.

A skin lightening serum followed by a skin lightening cream, both as described in composition embodiments above, were then applied to the subject's face. The subject's face was then massaged for about 20-minute. The skin temperature of the subject face increased about 1.5° F.

The skin lightening serum included the following ingredients: Aloe Barbadensis Leaf, Purified Water, Glycerin, Phospholipid, Arbutin, Linoleic Acid, Linolenic Acid, Tocopheryl Acetate, Ascorbyl Palmitate, Glutathione, Alcohol, Olea Europaea (Olive) Fruit Oil, Ethylhexyl Palmitate, Polyacrylamide, C13-14 Isoparaffin, Laureth-7, Hydrolyzed Beta Maritima Extract, Malus Domestica Fruit Cell Culture Extract, Xanthan Gum, Lecithin, Phenoxyethanol, Caprylyl Glycol, Glycyrrhiza Uralensis (Licorice) Root Extract, Caprylic/Capric Triglyceride, Hyaluronic Acid, Bellis Perennis (Daisy) Flower Extract, Salicyloyl Phytosphingosine, Simmondsia Chinensis (Jojoba) Seed Oil, Ceramide 3, Sphingolipids, Dimethyl Methoxyl Chromanyl Palmitate, and Fragrance(s).

The skin lightening cream included the following ingredients: Aloe Barbadensis Leaf, Purified Water, Glycerin, Lepidium Sativum Sprout Extract, Lecithin, Glycerin, Phenoxyethanol, Polyacrylamide, C13-14 Isoparaffin, Laureth-7, Olea Europaea (Olive) Fruit Oil, Ethylhexyl Palmitate, Arbutin, Caprylic/Capric Triglyceride, Malus Domestica Fruit Cell Culture Extract, Xanthan Gum, Lecithin, Hyaluronic Acid, Hydrolyzed Beta Maritima Extract, Butylene Glycol, Glycyrrhiza Uralensis (Licorice) Root Extract, Caprylyl Glycol, Tocopheryl Acetate, Bellis Perennis (Daisy) Flower Extract, Salicyloyl Phytosphingosine, Ceramide 3, and Fragrance.

A mask was then applied to the subjects face a left on for about 15 to 20 minutes. The mask was then removed with wet towels.

The toner above was then again sprayed on the subject's face to neutralize and remove any traces of the mask.

The lightening serum described above was then again applied to the subject's face.

As shown in FIGS. 1 and 2, the dramatic softening of the melasma blotch(es) after only one treatment with the composition described above. Additionally, the subject's skin had a healthy glow that looked hydrated, smoother and with a more consistent skin tone/color. Applying previously known skin care regimens alone, it would take weeks to see the results that occurred with the compositions described herein.

While applicant's disclosure has been provided with reference to specific embodiments above, it will be understood that modifications and alterations in the embodiments disclosed may be made by those practiced in the art without departing from the spirit and scope of the invention. All such modifications and alterations are intended to be covered.

What is claimed is:

1. A composition comprising:
a. two or more tyrosinase inhibiting compounds;
b. one or more extracts from at least one plant species to inhibit alpha-Melanoctye-stimulating hormone; and
c. one or more cosmetically acceptable carriers.

2. The composition as recited in claim 1, wherein the two or more tyrosinase inhibiting compounds comprises one or more peptides, a glycosylated hydroquinone, one or more anti-oxidants, or a combination thereof.

3. The composition as recited in claim 2, wherein the one or more peptides comprises Glutathione, Glutathione-Reductase, Dimethyl Methoxy Chromanyl Palmitate, or combinations thereof.

4. The composition as recited in claim 2, wherein the glycosylated hydroquinone comprises Arbutin.

5. The composition as recited in claim 2, wherein the one or more anti-oxidants comprises linolenic acid, tocopheryl Acetate, Olea Europaea fruit oil, Ascorbyl palmitate or a combination thereof.

7. The composition as recited in claim 1, wherein the one or more extracts comprises at least one of: Hydrolyzed Beta Maritima Extract or Lepidium Sativum Sprout Extract.

8. The composition as recited in claim 1, further comprising stem cells from at least one plant species.

9. The composition as recited in claim 1, wherein the one or more cosmetically acceptable carriers comprises one or more preservatives, one or more stabilizers, one or more humectants, one or more conditioners, one or more thickeners, one or more perfumes, one or more emollients, one or more moisturizers or a combination thereof.

10. The composition as recited in claim 1, wherein said composition is provided in a serum, cream, gel, emulsion, ointment, spray, powder, lotion or soap.

11. A method for reducing an appearance of age spots on skin by inhibiting melanin synthesis comprising applying the composition as recited in claim 1 to a portion of skin.

12. A cosmetic composition for topical application to a portion of skin comprising:
a. at least one peptide having tyrosinase inhibiting qualities;
b. at least on hydroquinone derivative;
c. at least one extract from at least one plant species; and
d. a cosmetically acceptable carrier.

13. The cosmetic composition of claim 12, wherein the peptide is selected from a group consisting: Glutathione, Glutathione-Reductase, Dimethyl Methoxy Chromanyl Palmitate or a combination thereof.

14. The cosmetic composition of claim 12, wherein the hydroquinone derivative comprises Arbutin.

15. The cosmetic composition of claim 12, wherein the at least one extract is selected from a group consisting: Hydrolyzed Beta Maritima Extract, Lepidium Sativum Sprout Extract, Glycyrrhiza Uralensis Root Extract, Bellis Perennis Flower Extract or a combination thereof.

16. The cosmetic composition of claim 12, further comprising at least one anti-oxidant selected from a group consisting: linolenic acid, tocopheryl Acetate, Olea Europaea fruit oil, Ascorbyl palmitate or a combinations thereof.

17. The cosmetic composition of claim 12, further comprising stem cells from at least one plant species.

18. The cosmetic composition of claim 12, wherein said composition is provided in a serum, cream, gel, emulsion, ointment, spray, powder or lotion.

19. The cosmetic composition of claim 12, wherein the cosmetically acceptable carrier comprises one or more preservatives, one or more stabilizers, one or more humectants, one or more thickeners, one or more conditioners, one or more perfumes, one or more emollients, one or more moisturizers or a combination thereof.

20. A method for inducing a depigmenting effect in melanocytes by inhibiting tyrosinase activity comprising applying the composition as recited in claim 12 to a portion of skin.
